(19) United States
(12) Patent Application Publication
(10) Pub. No.: US 2022/0117712 A1
(43) Pub. Date: Apr. 21, 2022

(54) Protective Device, in Particular for a Dental Surgeon

(71) Applicant: Select-Dental SA, Carouge (CH)

(72) Inventor: Charles-Henri Potel, Granges Veveyse (CH)

(21) Appl. No.: 17/071,650

(22) Filed: Oct. 15, 2020

(51) Int. Cl. A61C 19/00; A61B 90/40

(52) U.S. Cl. CPC A61C 19/00; A61B 90/40

(57) ABSTRACT

The invention relates to a protective device, which in particular prevents a dental surgeon from being contaminated by a patient during dental treatment device.

To be more specific, this protective device comprises:
a transparent shield (1);
a flexible support (3) connecting the transparent shield (1) to a chair (2),
the means for attaching (4,5) the flexible support (3) to the chair (2).

The invention pertains to the use of such a device.

The invention relates to a protective device, which in particular protects a dental surgeon from being contaminated by a patient during dental treatment, and to the use of such a device.

### BACKGROUND OF THE INVENTION

Appearance of the new corona 2019 virus caused a pandemic and forced the world to adopt new health rules and measures to limit or stop the spread of the virus. Many professionals therefore sought solutions to be better protected while being able to carry out their work as normally as possible.

### SUMMARY STATEMENT OF THE INVENTION

The main purpose of the invention is to enable a professional, such as a dental surgeon, to work as naturally as possible, regardless of whether his patient is infected with a virus, in particular the new corona 2019 virus. According to the invention, this purpose is achieved by means of a device according to the following point 1:

1.—Protective device, specifically intended for the dental surgeon, comprising:
a transparent shield;
a flexible support connecting the transparent shield to a chair, and
the means for attaching the flexible support to the chair. Advantageous features of the device in point 1 are described in the following points 2 to 9:

2.—Protective device according to point 1, in which the shield is substantially cone-shaped with the apex attached to the flexible support.

3.—Protective device according to point 1 or point 2, in which the apex of the cone formed by the shield is attached to the flexible support through a substantially toroidal part.

4.—Protective device according to point 3, in which the apex of the cone is truncated and the toroidal part has a groove to house this truncated apex.

5.—Protective device according to claim 4, in which the groove has an inclined lower circular wall.

6.—Protective device according to one of points 3 to 5, in which the toroidal shaped part is traversed by an opening extending along its axis of revolution.

7.—Protective device according to one of points 2 to 6, in which the base of the cone formed by the shield has petal-like parts extending, preferably evenly, around the periphery of the said base and projecting downwards.

8.—Protective device according to point 7, further comprising a protective sheet attached to the petals and extending downwards under the effect of gravity, thus forming a cylindrical extension of the shield's base.

9.—Device according to one of points 1 to 8, in which, in the dismantled state, the shield has a notch obtained by cutting out a piece of cone defined by connecting the two sides of an arc of the cone's base to the apex of this cone, and in which the two rectilinear edges of the notch have means to be attached to each other.

According to another feature, the invention is also intended to be used in accordance with the following point 10:

10.—Use of a device according to point 6 or according to one of the points 7 to 9 linked to point 6, in which a suction hose is connected to the opening of the toroidal shaped part and suction is started.

More features and advantages of the invention will now be described in detail in the following statement given with reference to the attached figures, which schematically represent:

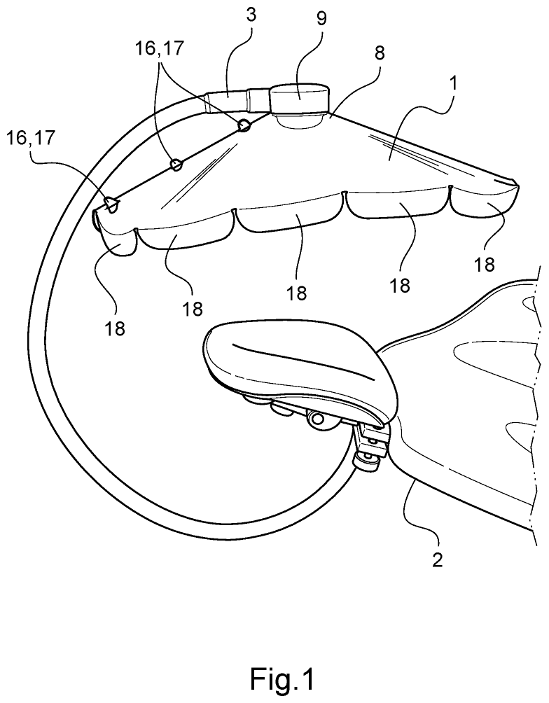

FIG. 1: front view of a protective device according to the invention;

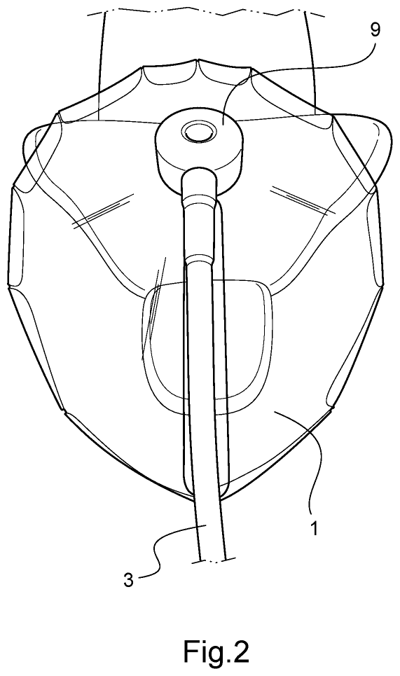

FIG. 2: top view of the device in FIG. 1;

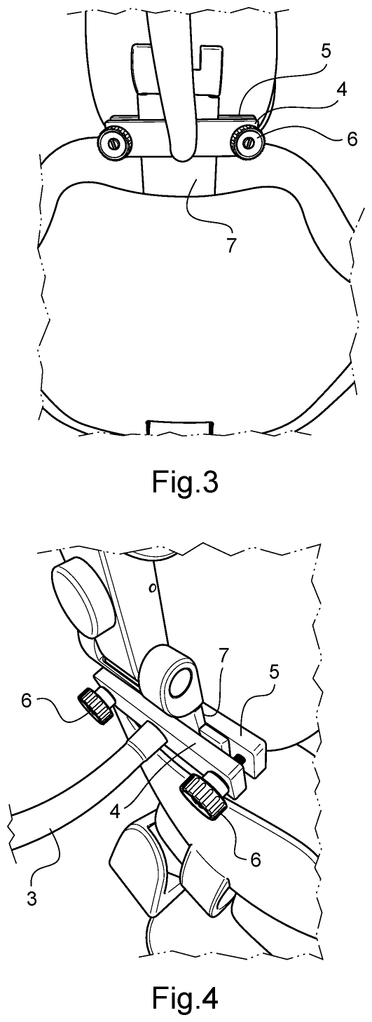

FIG. 3 and FIG. 4: a method that can be used to attach the device according to the invention to a dental surgeon's chair;

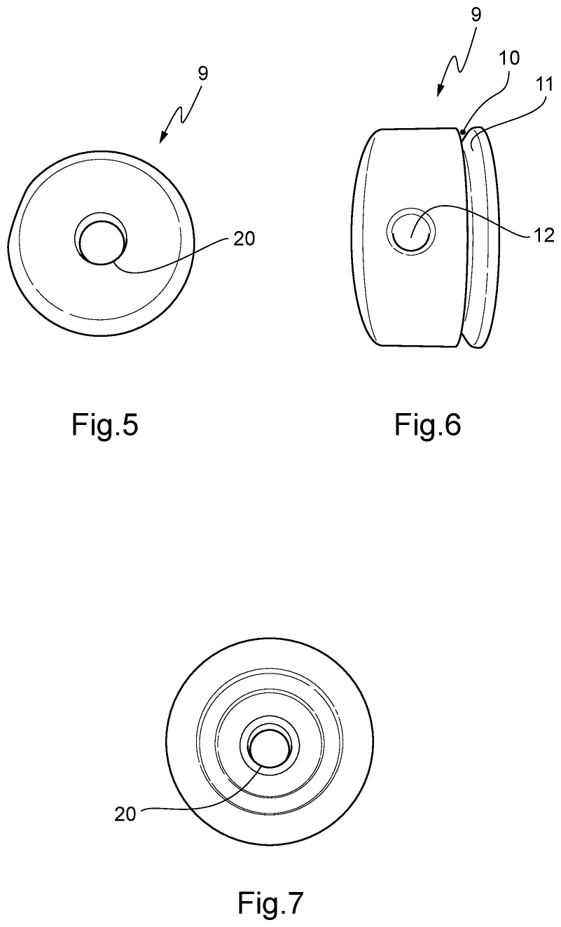

FIG. 5, FIG. 6 and FIG. 7: a toroidal shape part of a device according to the invention, its top view, front view and bottom view;

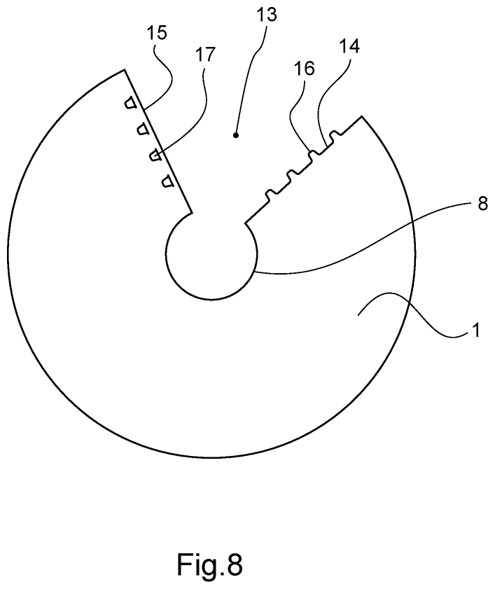

FIG. 8: a variant of a shield of a device according to the invention, in dismantled state;

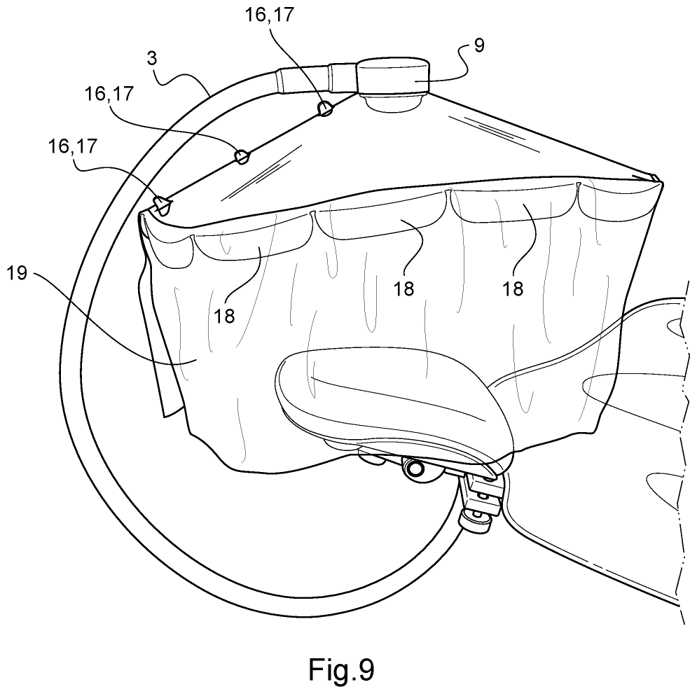

FIG. 9: an advantageous variant of the device according to the invention; and

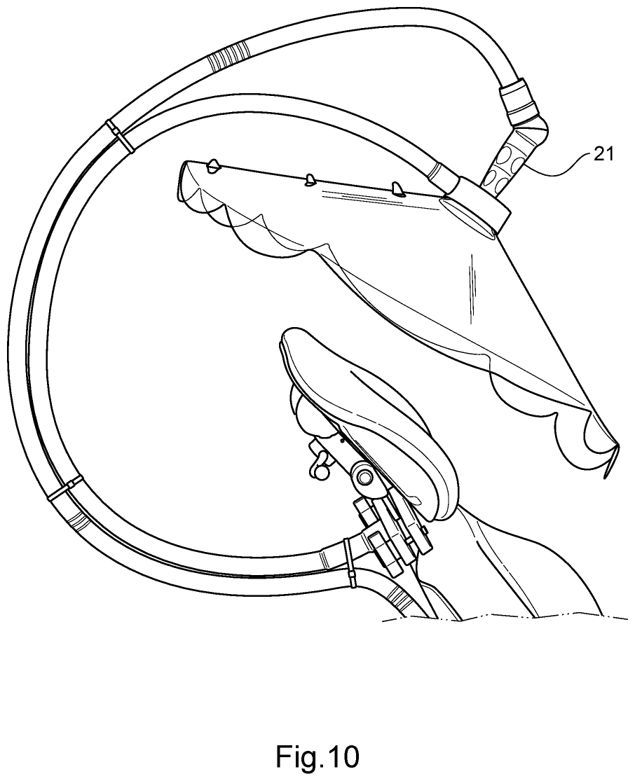

FIG. 10: an example of usage of a device according to the invention.

### DETAILED DESCRIPTION OF THE INVENTION

An example the device's embodiment according to the invention is shown in FIGS. 1 to 7.

As can be seen in FIG. 1, the device first comprises a shield 1, which is transparent in order to allow the dentist to examine and work inside the patient's mouth.

Preferably, this shield 1 is significantly cone-shaped and is attached by its apex 8 to a support 3 which connects it to a dentist's chair 2.

Support 3 is manually flexible so that the dentist can position it as best as possible above the patient's head and it remains in the position given to it by the dentist, as can also be seen in FIG. 2.

For example, the support 3 can be shaped like a tube with an internal metal frame, which gives it its flexibility, and at its lower end it is equipped with fixtures, visible in FIGS. 3 and 4, with which it can be attached to the chair 2. These fixtures include, for example, two bars 4 and 5 intended to clamp between them, with the help of screw 6, a part 7 of the chair's 2 backrest.

Advantageously, the shield 1 is attached to the flexible support 3 by means of a substantially toroidal shaped part 9 visible in FIGS. 1 and 2 and in more detail in FIGS. 5 to 7.

Apex 8 of the cone formed by the shield 1 is then preferably truncated, so that its edge can be housed in a groove 10 (see FIG. 6) whose lower circular wall 11 is preferably inclined downwards to match the truncated cone shape of the shield 1.

A hole 12 can be provided to house the upper end of the flexible support 3.

It is desirable that the shield 1, in its disassembled state shown in FIG. 8, have a notch 13 obtained by cutting out a piece of cone defined by connecting the two sides of an arc of the cone's base to the apex of this cone. In order to be able to close the truncated cone, the two rectilinear edges 14 and 15 of the notch thus comprise means of attachment 16,17, such as lugs 16 and holes 17, helping on the one hand, to hold one edge slightly superimposed on the other edge, aimed at achieving watertightness, and, on the other hand, to close the edge of the apex 8 around the groove 10 so as to secure the shield 1 to the toroidal part 9, preferably also in a watertight manner. The apex 8 of the shield 1 is then supported by the inclined circular wall 11. The attachment of one edge on the other is reversible, which enables the dentist to remove the shield after the patient has left and replace a clean one for the next patient.

The used shield can then be discarded or disinfected to be reused.

In an advantageous embodiment of the invention, the base of the cone formed by the shield comprises petal-shaped parts 18 extending around the periphery of the said base and projecting downwards. As can be seen in FIG. 9, these petals 18 can be used to attach a flexible, preferably transparent protective sheet 19 which extends downwards under the effect of gravity and thus forms a cylindrical extension of the shield's 1 base.

Thus, thanks to its flexibility, the protective sheet 19 can be lifted to allow the dentist's hands and arms to pass through. It does not interfere with the dentist's work and at the same time protects him or her from splashes, especially when scaling.

Attachment of the protective sheet 19 to the shield is reversible, which allows the dentist to remove it, just like the shield after the patient has left, and to replace a clean protective sheet for the next patient.

The used protective sheet can then be discarded or disinfected to be reused.

In another embodiment of the invention visible in FIGS. 5 and 7, the toroidal-shaped part 9 has an opening 20 which runs through it along its axis of revolution.

This opening 20 allows connecting a suction hose 21 visible in FIG. 10.

In this case it is advantageous to use a suction hose 21 connected to the suction system of the dentist's apparatus. If suctioning is not required, the suction hose 21 can be removed and the opening 20 closed with a cap (not shown). The parts constituting the device according to the invention may be made of known commercially available materials.

1. Protective device, specifically intended for a dental surgeon, comprising:
a transparent shield (1);
a flexible support (3) connecting the transparent shield to a chair and
the means for attaching (4,5) the flexible support (3) to the chair (2).

2. Protective device according to claim 1, in which the transparent shield (1) is substantially cone-shaped with the apex (8) attached to the flexible support (3).

3. Protective device according to claim 2, in which the apex (8) of the cone formed by the transparent shield (1) is attached to the flexible support (3) through a substantially toroidal part (9).

4. Protective device according to claim 3, in which the apex (8) of the cone is truncated and the toroidal part (9) has a groove (10) to house this truncated apex (8).

5. Protective device according to claim 4, in which the groove (10) has an inclined lower (11) circular wall.

6. Protective device according to claim 3, in which the toroidal shaped part (9) is traversed by an opening (20) extending along its axis of revolution.

7. Protective device according to claim 4, in which the toroidal shaped part (9) is traversed by an opening (20) extending along its axis of revolution.

8. Protective device according to claim 5, in which the toroidal shaped part (9) is traversed by an opening (20) extending along its axis of revolution.

9. Protective device according to claim 2, in which the base of the cone formed by the transparent shield (1) has petal-like (18) parts extending around the periphery of the said base and projecting downwards.

10. Protective device according to claim 9, further comprising a protective sheet (19) attached to the petals (18) and extending downwards under the effect of gravity, thus forming a cylindrical extension of the transparent shield's (1) base.

11. Device according to claim 2, in which, in the dismantled state, the transparent shield (1) has a notch (13) obtained by cutting out a piece of cone defined by connecting the two sides of an arc of the cone's base to the apex (8) of this cone, and in which the two rectilinear edges (14,15) of the notch (13) are, in the mounted state, fixed one (14) to the other (15) through means of attachment (16,17).

12. Device according to claim 3, in which, in the dismantled state, the transparent shield (1) has a notch (13) obtained by cutting out a piece of cone defined by connecting the two sides of an arc of the cone's base to the apex (8) of this cone, and in which the two rectilinear edges (14,15) of the notch (13) are, in the mounted state, fixed one (14) to the other (15) through means of attachment (16,17).

13. Device according to claim 4, in which, in the dismantled state, the transparent shield (1) has a notch (13) obtained by cutting out a piece of cone defined by connecting the two sides of an arc of the cone's base to the apex (8) of this cone, and in which the two rectilinear edges (14,15) of the notch (13) are, in the mounted state, fixed one (14) to the other (15) through means of attachment (16,17).

14. Device according to claim 5, in which, in the dismantled state, the transparent shield (1) has a notch (13) obtained by cutting out a piece of cone defined by connecting the two sides of an arc of the cone's base to the apex (8) of this cone, and in which the two rectilinear edges (14,15) of the notch (13) are, in the mounted state, fixed one (14) to the other (15) through means of attachment (16,17).

15. Device according to claim 6, in which, in the dismantled state, the transparent shield (1) has a notch (13) obtained by cutting out a piece of cone defined by connecting the two sides of an arc of the cone's base to the apex (8) of this cone, and in which the two rectilinear edges (14,15) of the notch (13) are, in the mounted state, fixed one (14) to the other (15) through means of attachment (16,17).

16. Device according to claim 7, in which, in the dismantled state, the transparent shield (1) has a notch (13) obtained by cutting out a piece of cone defined by connecting the two sides of an arc of the cone's base to the apex (8) of this cone, and in which the two rectilinear edges (14,15) of the notch (13) are, in the mounted state, fixed one (14) to the other (15) through means of attachment (16,17).

17. Device according to claim 8, in which, in the dismantled state, the transparent shield (1) has a notch (13) obtained by cutting out a piece of cone defined by connecting the two sides of an arc of the cone's base to the apex (8) of this cone, and in which the two rectilinear edges (14,15) of the notch (13) are, in the mounted state, fixed one (14) to the other (15) through means of attachment (16,17).

18. Device according to claim 9, in which, in the dismantled state, the transparent shield (1) has a notch (13) obtained by cutting out a piece of cone defined by connecting the two sides of an arc of the cone's base to the apex (8) of this cone, and in which the two rectilinear edges (14,15) of the notch (13) are, in the mounted state, fixed one (14) to the other (15) through means of attachment (16,17).

19. Device according to claim 6, in which the opening (20) is closed with a cap.

20. Device according to claim 7, in which the opening (20) is closed with a cap.
